(19) United States
(12) Patent Application Publication
(10) Pub. No.: US 2017/0273935 A1
(43) Pub. Date: Sep. 28, 2017

(54) CINEOLE-CONTAINING COMPOSITION FOR NASAL APPLICATION

(71) Applicant: Maria Clementine Martin Klosterfrau Vertriebsgesellschaft mbH, Köln (DE)

(72) Inventor: Markus UNKAUF, München (DE)

(21) Appl. No.: 15/504,151

(22) PCT Filed: Jun. 25, 2015
(86) PCT No.: PCT/EP2015/064373

§ 371 Date: Feb. 15, 2017

(30) Foreign Application Priority Data
Aug. 18, 2014 (DE) 10 2014 012 040.6
Aug. 22, 2014 (DE) 10 2014 012 278.6
Sep. 10, 2014 (DE) 10 2014 013 145.9
Sep. 11, 2014 (DE) 10 2014 013 189.0
Nov. 19, 2014 (DE) 10 2014 116 903.4

(51) Int. Cl. A61K 31/34; A61K 9/00; A61K 31/164; A61K 47/10; A61K 31/197; A61K 47/02; A61K 47/44; A61K 47/18; A61K 9/127; A61K 31/505

(52) U.S. Cl. CPC A61K 31/34; A61K 9/127; A61K 9/0043; A61K 31/164; A61K 31/505; A61K 31/197; A61K 47/02; A61K 47/44; A61K 47/186; A61K 47/10

(57) ABSTRACT

The invention relates to a pharmaceutical composition containing cineole for topical, in particular nasal, preferably intranasal application, to the use thereof and to an application device containing said pharmaceutical composition.

### CROSS-REFERENCES TO RELATED APPLICATIONS

This application is a National Stage filing of International Application PCT/EP 2015/064373, filed Jun. 25, 2015, entitled CINEOLE-CONTAINING COMPOSITION FOR NASAL APPLICATION, claiming priority to DE 10 2014 012 040.6 filed Aug. 18, 2014, to DE 10 2014 012 278.6 filed Aug. 22, 2014, to DE 10 2014 013 145.9 filed Sep. 10, 2014, to DE 10 2014 013 189.0 filed Sep. 11, 2014 and to DE 10 2014 116 903.4 filed Nov. 19, 2014. The subject application claims priority to PCT/EP 2015/064373, and to DE 10 2014 012 040.6, DE 10 2014 012 278.6, DE 10 2014 013 145.9, DE 10 2014 013 189.0, DE 10 2014 116 903.4 and incorporates all by reference herein, in their entirety.

### BACKGROUND OF THE INVENTION

The present invention relates to the technical (i.e., medical and therapeutic) field of treating rhinitises.

More particularly, the present invention relates to a cineole-containing composition, especially a cineole-containing pharmaceutical composition, which is preferably suitable for topical application, especially nasal application, preferably intranasal application, especially for the treatment of rhinitises, and to the use of said cineole-containing composition and to an applicator containing said cineole-containing composition. Hereinafter, the cineole-containing composition according to the invention will occasionally also be synonymously referred to as inventive combination or inventive active-ingredient combination or as inventive antirhinitis agent (antirhinitic).

Within the scope of the present invention, a rhinitis (occasionally also synonymously referred to as nasal catarrh, cold or coryza) is understood to mean in particular an acute or chronic inflammation of the nasal mucous membranes, it being possible for the rhinitis to be in particular of infectious (e.g., viral or bacterial), allergic or pseudoallergic origin. Most commonly, a rhinitis occurs as part of a so-called cold.

Besides a distinction between acute rhinitis on the one hand and chronic rhinitis on the other, a distinction is also made between various forms of rhinitises, for example the following rhinitis forms: Rhinitis acuta, Rhinitis atrophicans, Rhinitis allergica, Rhinitis hypertrophica, Rhinitis medicamentosa, Rhinitis pseudomembranacea, Rhinitis sicca, Rhinitis vasomotorica and environmental rhinitis.

In general, the so-called acute rhinitis (Rhinitis acuta), i.e., the common cold, is a generally harmless infection of the nasal mucous membranes and thus an infectious rhinitis, which can usually be triggered virally, especially by a multiplicity of viruses (especially rhinoviruses and/or adenoviruses), but occasionally also bacterially. The main feature of an acute rhinitis is a so-called runny nose and a congestion of the nose as a result of the swelling of the mucous membranes.

Altogether, more than 200 “cold viruses” are known as possible triggers of a viral rhinitis, as can usually occur as part of a common cold. However, during a cold, which generally starts with a rhinitis, the rhinitis acuta generally disappears. Occasionally, a chronification can, however, sometimes also occur, which is frequently associated with an increase in volume of the mucous membranes especially in the region of the turbinates with obstruction of nasal breathing.

A rhinitis (i.e., an inflammation of the nasal mucous membrane), especially an acute rhinitis, can frequently also be simultaneously present with an inflammation of the mucous membrane of the sinuses (sinusitis). A rhinosinusitis is also spoken of in such a case.

For further details relating to the term rhinitis, reference can be made in particular to Pschyrembel, Medizinisches Wörterbuch [Medical dictionary], 257th edition, pages 1331/1332, in particular the keywords: “Rhinitis”, “Rhinitis allergica”, “Rhinitis atrophicans”, “Rhinitis hyperplastica”, “Rhinitis pseudomembranacea”, “Rhinitis sicca” and “Rhinitis vasomotorica”.

Thus, since there is a multiplicity of different types of viruses which can trigger an acute viral rhinitis and since there is a multiplicity of causes of the occurrence of a rhinitis, rhinitises, especially acute rhinitises, generally cannot be treated causally, but instead only symptomatically, especially topically.

For this purpose, use is made in most cases of compositions to be applied intranasally, containing so-called sympathomimetics (also synonymously referred to as “swelling reducers”, “decongestants” or the like), preferably alpha sympathomimetics (such as, for example, xylometazoline and oxymetazoline or the physiologically tolerable or safe salts thereof).

Although said sympathomimetics initially lead, owing to their vasoconstrictive properties after local or topical application in the nose, to a reduced swelling of nasal mucous membranes, they frequently bring about, in the case of repeated application, a drying-up of the nasal mucous membranes, associated with inflammatory irritations of the nasal mucous membranes (and this frequently leads to an increased risk of infection, since the nasal mucous membrane in the dried-up and inflamed state can no longer fully maintain its protective function and filter function and consequently pathogens can reach the airways unhindered).

Furthermore, the sympathomimetics act neither antibacterially nor antivirally. Also, the sympathomimetics sometimes even have severe systemic (e.g., cardiovascular) adverse effects, especially in the case of overdosage or in the case of the therapy of certain patients (e.g., infants, patients with preexisting cardiovascular conditions, etc.).

To counteract at least in part the numerous adverse effects of the sympathomimetics and to improve at least in some way the in some cases inadequate activity profile of the sympathomimetics in the case of the treatment of rhinitises, further active ingredients or further ingredients are sometimes incorporated in sympathomimetic-containing compositions intended for intranasal application, for example plant extracts or plant ingredients, moisturizing or mucous-membrane-caring additives, antihistamines, salts, etc. (cf. for example DE 195 41 919 A1, DE 195 49 421 A1 and DE 103 56 248 A1).

Furthermore, sympathomimetic-free compositions intended for intranasal application are also known from the prior art for the treatment of rhinitises, which compositions, however, frequently have insufficient efficacy with respect to an efficient treatment of rhinitises.

### BRIEF SUMMARY OF THE INVENTION

The problem addressed by the present invention is therefore that of providing a composition, especially a pharmaceutical composition, which is suitable for topical application, especially nasal application, preferably intranasal application, especially for the treatment of rhinitises, and which at least largely avoids or else at least attenuates the above-described disadvantages of the prior art.

More particularly, it is intended that such a composition have an improved efficiency of action and/or an improved adverse effect profile in comparison with conventional sympathomimetic-containing pharmaceutical preparations intended for the treatment of rhinitises by means of topical or intranasal application.

To solve the above-described problem, the present invention proposes—according to a first aspect of the present invention—a cineole-containing composition, especially a pharmaceutical composition, as claimed in claim 1; further, especially advantageous designs of the composition according to the invention are the subject matter of the relevant dependent claims.

The present invention further provides—according to a second aspect of the present invention—an applicator containing the cineole-containing composition according to the invention as claimed in the relevant device claim.

Lastly, the present invention provides for—according to a further and third aspect of the present invention—the inventive use of the cineole-containing composition according to the present invention, as defined in the corresponding use claims.

It is self-evident in the following explanations that designs, embodiments, advantages and the like which are explained below only in relation to one aspect of the invention in order to avoid repetition also, self-evidently, apply accordingly to the other aspects of the invention without this needing a separate mention.

In all relative or percentage weight-based data, especially specified quantities, mentioned below, it should be further noted that these are to be selected by a person skilled in the art within the scope of the present invention such that they always make up or add up to 100% or 100% by weight in total with inclusion of all components or ingredients, especially as defined below; however, this is self-evident to a person skilled in the art.

Apart from that, a person skilled in the art can—depending on the application or depending on the individual case—deviate from the specified weights, specified quantities and specified ranges that are stated below without departing from the scope of the present invention.

Moreover, all specified values or specified parameters or the like that are mentioned below can absolutely be ascertained or determined using normed/standardized or explicitly specified determination methods or else using determination or measurement methods familiar per se to a person skilled in the art in this field.

With this said, the present invention will now be elucidated in detail below.

### DETAILED DESCRIPTION OF THE INVENTION

The present invention therefore provides—according to a first aspect of the present invention—a cineole-containing composition, especially a pharmaceutical composition, which is preferably suitable for topical application, especially nasal application, preferably intranasal application, especially for the treatment of rhinitises,

wherein the composition contains, in combination and in effective, especially pharmaceutically effective, quantities in each case,
(a) cineole, preferably 1,8-cineole;
and
(b) (b1) pantothenol, preferably dexpanthenol (D-pantothenol), or the physiologically safe esters thereof and/or
   (b2) pantothenic acid or the physiologically safe salts thereof.

The present invention is associated with numerous advantages and characteristics which are discussed below in a nonrestricting manner and are to be judged as evidence of patentability.

Within the scope of the present invention, an efficiently acting antirhinitis agent (antirhinitic) is provided which manages without the mandatory presence of alpha sympathomimetics, making it possible to avoid the undesired adverse effects of alpha sympathomimetics.

Owing to the presence of component (a), i.e., of the cineole, especially in the form of 1,8-cineole, the composition according to the invention has, in its intended use, not only an antibacterial (i.e., bactericidal or bacteriostatic) effect, but additionally—as found by the applicant, completely surprisingly—also an antiviral (i.e., virucidal or virustatic) effect.

Furthermore, the composition according to the invention acts in an inflammation-inhibiting or anti-inflammatory manner and moreover in a secretolytic or expectorant manner. In this way, the composition according to the invention can efficiently counteract inflammations of the nasal mucuous membrane (as always occur in rhinitises). Moreover, the composition according to the invention brings about a good moisturization of the nasal mucuous membrane.

Owing to the synergistic cooperation of components (a) and (b), an antirhinitic effect is achieved overall. More particularly, there is efficient counteraction of the symptoms of rhinitises, especially also the so-called runny nose (rhinorrhea) (i.e., the composition or combination according to the invention also has an antirhinorrheic activity profile or activity potential).

Also, the composition according to the invention efficiently counteracts the obstruction of nasal breathing which occurs in rhinitises.

However, the composition according to the invention does not only act in an inflammation-inhibiting or anti-inflammatory manner, but also has, especially owing to the presence of component (b), a mucous-membrane-protective and wound-healing-promoting effect, and so there is efficient counteraction of inflammations of the nasal mucous membrane, as always occur in rhinitises. Moreover, component (b) acts to the effect that it cares for and moisturizes the nasal mucous membrane, and so in this way there is also counteraction of the drying-up of the nasal mucous membrane and of scab formation.

Especially owing to the antimicrobial, especially bactericidal or bacteriostatic, activity potential of the cineole, a good stability, especially storage stability and long-term stability, of the composition according to the invention is also achieved. By means of additional measures (e.g., pH adjustment or pH control and also use of chemical buffer systems, emulsifiers, thickeners, etc.), it is possible to further improve the stability of the composition according to the invention.

By means of optional incorporation of further active ingredients and/or further ingredients, it is possible to control or specifically adjust activity profile and stability behavior of the composition according to the invention.

As explained above, the cineole-containing composition according to the present invention contains cineole, preferably 1,8-cineole, as component (a).

With regard to the active ingredient cineole, especially 1,8-cineole, the following can be stated in relation thereto: Cineole, especially in the form of 1,8-cineole, is part of the bicyclic epoxy monoterpenes, more precisely the limonene oxides. Synonymous names for 1,8-cineole having the molecular formula C₁₀H₁₈O are eucalyptol, limonene 1,8-oxide, 1,8-epoxy-p-menthane or 1,3,3-trimethyl-2-oxabicyclo[2.2.2]octane. It is a colorless liquid with a spicy, camphor-like odor having a melting point of +1.5° C. and a boiling point of from 176 to 177° C., which liquid is insoluble in water, but miscible with most organic solvents. Naturally, 1,8-cineole occurs as the main constituent of the eucalyptus oil (eucalyptus oil contains up to 85% by weight of 1,8-cineole), but also in other plants, for example in mint, common sage, thyme, basil and tea tree; furthermore, 1,8-cineole is, for example, present in niaouli oil, juniper oil, piper oil, cannabis oil, cajeput oil, sage oil, myrtle oil and other essential oils. Technical-grade 1,8-cineole, which is generally from 99.6% to 99.8%, is generally obtained by fractional distillation of eucalyptus oil.

For further details relating to the active ingredient 1,8-cineole, reference can, for example, be made to RÖMPP Chemielexikon [RÖMPP chemical dictionary], Georg Thieme Verlag, Stuttgart/New York, 10th edition, volume 1, 1996, page 752, keyword: “Cineole”, and to the literature referenced therein.

As already explained above, the cineole, especially 1,8-cineole, in the composition according to the invention develops, in the intended use thereof, not only an antibacterial (i.e., bactericidal or bacteriostatic) effect, but additionally (as found by the applicant, completely surprisingly) also an antiviral (i.e., virucidal or virustatic) effect. Furthermore, the cineole, especially 1,8-cineole, within the scope of the composition according to the invention acts altogether in an inflammation-inhibiting or anti-inflammatory manner and moreover in a secretolytic or expectorant manner; in this way, it efficiently counteracts in synergistic combination with component (b) (i.e., pantothenol or pantothenic acid)—inflammations of the nasal mucous membrane and moreover ensures a good moisturization of the nasal mucous membrane. In synergistic cooperation with component (b), an antirhinitic effect is achieved overall and moreover there is efficient counteraction of the symptoms of rhinitises, especially also the so-called runny nose (rhinorrhea) (i.e., the composition or combination according to the invention also has an antirhinorrheic activity profile or activity potential).

Also, the cineole in cooperation with component (b) efficiently counteracts the obstruction of nasal breathing which occurs in rhinitises. Owing to its antimicrobial, especially bactericidal or bacteriostatic, activity potential, the cineole is also beneficial with regard to the stability, especially storage stability and long-term stability, of the composition according to the invention.

According to the invention, it is preferred when component (a) contains the cineole as pure substance, in particular free of other terpenes, by preference with a purity of at least 95% by weight, especially at least 96% by weight, preferably at least 97% by weight, particularly preferably at least 98% by weight, very particularly preferably at least 99% by weight, even more preferably at least 99.5%, based on the cineole. This means that the composition preferably contains no further terpene apart from the cineole.

In other words, the cineole is present as pure substance in the cineole-containing composition according to the present invention, i.e., the cineole used in the cineole-containing composition according to the present invention is free of other terpenes or contains no other terpenes.

Preferably, the cineole used as component (a) has a purity of at least 95% by weight, especially at least 96% by weight, preferably at least 97% by weight, particularly preferably at least 98% by weight, very particularly preferably at least 99% by weight, even more preferably at least 99.5%, based on the cineole.

According to a particular embodiment of the present invention, the composition according to the invention contains the cineole as 1,8-cineole or the cineole is present as 1,8-cineole (i.e., in other words, according to this particular embodiment, component (a) contains the cineole in the form of 1,8-cineole).

The use of cineole as pure substance, especially in the form of 1,8-cineole, ensures a defined or controllable activity profile. More particularly, undesired or unpredictable interactions and adverse effects are avoided owing to the absence of further terpenes.

According to a particular embodiment of the present invention, component (a) or the cineole can be present or formulated in a liposome-surrounded and/or liposomally packaged form. This achieves in particular an improved solubility or emulsifiability of the cineole with, at the same time, good stabilization of the cineole in the composition and with good bioavailability.

The quantity of component (a) or cineole in the cineole-containing composition according to the present invention can vary or be adjusted within wide ranges. More particularly, it can be envisaged that the composition according to the invention contains component (a) or the cineole, based on the composition, in relative quantities within the range from 0.001 to 10% by weight, especially from 0.002 to 5% by weight, by preference from 0.01 to 3% by weight, preferably from 0.05 to 3% by weight, particularly preferably from 0.1 to 2.5% by weight, very particularly preferably from 0.2 to 2% by weight, even more preferably from 0.3 to 1% by weight, most preferably from 0.4 to 0.8% by weight. Nevertheless, it may be necessary, depending on the individual case or depending on the application, to deviate from the aforementioned values without departing from the scope of the present invention. This is at the discretion of a person skilled in the art.

In connection with the application or administration of component (a) (i.e., cineole, preferably in the form of 1,8-cineole), it is preferred according to the invention when component (a) is administered with an individual dose within the range from 0.01 mg to 50 mg, especially within the range from 0.1 mg to 25 mg, by preference within the range from 0.5 mg to 20 mg, preferably within the range from 1 mg to 15 mg, particularly preferably within the range from 2 mg to 10 mg, or when component (a) is prepared for administration with an individual dose within the range from 0.01 mg to 50 mg, especially within the range from 0.1 mg to 25 mg, by preference within the range from 0.5 mg to 20 mg, preferably within the range from 1 mg to 15 mg, particularly preferably within the range from 2 mg to 10 mg. With regard to the aforementioned individual doses, it is moreover customary according to the invention that from 2 to 10 individual doses per day, especially from 3 to 5 individual doses per day, are administered, preferably distributed throughout the day, or that component (a) is prepared for administration in from 2 to 10 individual doses per day, especially from 3 to 5 individual doses per day, preferably distributed throughout the day.

With regard to component (b) of the composition according to the invention, said component (b) can—as already explained above—comprise (b1) pantothenol, preferably dexpanthenol (D-pantothenol), or the physiologically safe esters thereof and/or (b2) pantothenic acid or the physiologically safe salts thereof (pantothenates).

As explained above, the cineole-containing composition according to the present invention thus contains, as component (b), pantothenol, preferably dexpanthenol (D-pantothenol), or the physiologically safe esters thereof and/or pantothenic acid or the physiologically safe salts thereof. The active ingredient pantothenol is, from a chemical point of view, part of the polyols and amides and is, from a physiological point of view, a provitamin (i.e., a precursor of a vitamin of the vitamin B group) which is converted in the body to pantothenic acid (vitamin B5). The active ingredient pantothenic acid in turn is a constituent of the coenzyme A and thus plays a major role in (mucous) membrane metabolism.

As already explained above, component (b) (i.e., pantothenol, preferably dexpanthenol, or the physiologically safe esters thereof or pantothenic acid or the physiologically safe salts thereof) in the composition according to the invention develops, in the intended use thereof, not only a mucous-membrane-protective and wound-healing-promoting effect, but also acts independently in an inflammation-inhibiting or anti-inflammatory manner. In synergistic combination with the cineole, especially 1,8-cineole, there is consequently efficient counteraction of inflammations of the nasal mucous membrane, as always occur in rhinitises. Moreover, component (b) acts on the nasal mucous membrane in a caring and moisturizing manner, and so in this way a good moisturization of the nasal mucous membrane is brought about or there is counteraction of a drying-up of the nasal mucous membrane and of scab formation. Also, component (b) in cooperation with component (a) achieves an improved nasal breathing. In synergistic cooperation with component (a), an antirhinitic effect is consequently achieved overall and moreover there is efficient counteraction of the symptoms of rhinitises, especially also runny nose (rhinorrhea) (i.e., the composition or combination according to the invention also has—as stated above—an antirhinorrheic activity profile or activity potential).

According to the invention, it is preferred when the composition contains, as component (b), pantothenol, preferably dexpanthenol (D-pantothenol), or the physiologically safe esters thereof.

According to the invention, it is particularly preferred when the composition contains, as component (b), pantothenol, preferably in the form of dexpanthenol (D-pantothenol).

The quantity of component (b) in the cineole-containing composition according to the present invention can vary or be adjusted within wide ranges. More particularly, it can be envisaged that the composition according to the invention contains component (b), based on the composition, in relative quantities within the range from 0.01 to 10% by weight, especially from 0.1 to 8% by weight, by preference from 0.5 to 7% by weight, preferably from 1 to 6.5% by weight, particularly preferably from 2 to 6% by weight, very particularly preferably from 4 to 6% by weight, even more preferably from 4.5 to 5.5% by weight. Nevertheless, it may be necessary, depending on the individual case or depending on the application, to deviate from the aforementioned values without departing from the scope of the present invention. This is at the discretion of a person skilled in the art.

In connection with the application or administration of component (b) (i.e., pantothenol or pantothenic acid), it is preferred according to the invention when component (b), preferably in the form of pantothenol, particularly preferably dexpanthenol, is administered with an individual dose within the range from 0.1 mg to 50 mg, especially within the range from 0.5 mg to 25 mg, by preference within the range from 1 mg to 20 mg, preferably within the range from 2.5 mg to 10 mg, particularly preferably within the range from 4 mg to 8 mg, or when component (b), preferably in the form of pantothenol, particularly preferably dexpanthenol, is prepared for administration with an individual dose within the range from 0.1 mg to 50 mg, especially within the range from 0.5 mg to 25 mg, by preference within the range from 1 mg to 20 mg, preferably within the range from 2.5 mg to 10 mg, particularly preferably within the range from 4 mg to 8 mg. With regard to the aforementioned individual doses, it is moreover customary according to the invention that from 2 to 10 individual doses per day, especially from 3 to 5 individual doses per day, are administered, preferably distributed throughout the day, or that component (b), preferably in the form of pantothenol, particularly preferably dexpanthenol, is prepared for administration in from 2 to 10 individual doses per day, especially from 3 to 5 individual doses per day, preferably distributed throughout the day.

For the efficacy of components (a) and (b) in the composition according to the invention, the quantity ratio thereof is also of importance. According to one embodiment preferred according to the invention, it can be envisaged that the composition according to the invention contains components (a) and (b) in a quantity ratio of component (a) to component (b) within the range from 1:1 to 1:500, especially from 1:1.25 to 1:100, by preference from 1:1.5 to 1:75, preferably from 1:2 to 1:50, particularly preferably from 1:2.5 to 1:25, very particularly preferably from 1:2.75 to 1:20, even more preferably from 1:3 to 1:10. Nevertheless, it may be necessary, depending on the individual case or depending on the application, to deviate from the aforementioned values without departing from the scope of the present invention (and this is at the discretion of a person skilled in the art).

According to a particular embodiment of the present invention, it can be envisaged that the composition according to the present invention also contains at least one emulsifier as component (c).

An emulsifier in the context of the present invention is a formulation excipient which serves to mix and to stabilize two substances or liquids which are not miscible with one another or only minimally miscible with one another to form a finely distributed mixture (i.e., emulsion). Within the scope of the present invention, it is intended in particular that the miscibility or solubility of component (a), i.e., of the cineole, but possibly also of component (b), with or in the aqueous excipient (vehicle) of the composition according to the invention be improved and the resulting emulsion be stabilized. However, in this way, not only is the miscibility or emulsifiability of the active ingredients in the water-based excipient improved and the stability of the resulting composition according to the invention increased, but the bioavailability of the active ingredients, especially of the cineole, is also improved.

The use of an emulsifier with respect to the composition according to the invention is absolutely optional, but is a preferred embodiment for the aforementioned reasons.

The quantity of component (c) or emulsifier in the cineole-containing composition according to the present invention can vary or be adjusted within wide ranges. More particularly, it can be envisaged that the composition according to the invention contains component (c) and/or the emulsifier, based on the composition, in relative quantities within the range from 0.0001 to 10% by weight, especially from 0.001 to 5% by weight, by preference from 0.005 to 4% by weight, preferably from 0.01 to 3% by weight, particularly preferably from 0.05 to 2.5% by weight, very particularly preferably from 0.1 to 2% by weight, even more preferably from 0.2 to 1.8% by weight, most preferably from 0.5 to 1.5% by weight.
contains from 0.02 to 0.08% by weight. Nevertheless, it may be necessary, depending on the individual case or depending on the application, to deviate from the aforementioned values without departing from the scope of the present invention. This is at the discretion of a person skilled in the art.

Component (c) or the emulsifier can absolutely be selected from a multiplicity of diverse compounds. According to a preferred embodiment, it can be envisaged that component (c) or the emulsifier is selected from the group of (i) fatty acids, fatty acid salts, fatty acid amides and fatty acid esters, especially fatty acid esters, (ii) optionally (poly)ethoxylated and/or hydrogenated (i.e., partially or fully hydrogenated) ricinus oils (=castor oils), (iii) organic carboxylic acids and the salts and esters thereof, especially citric acid and the salts thereof (citrates), (iv) organic betaines, (v) organic ammonium compounds, (vi) physiologically safe ionic or nonionic surfactants, (vii) phospholipids, especially phosphatidylcholines and lecithins, (viii) poloxamers (ethylene oxide-propylene oxide block copolymers), (ix) esters of polyhydric alcohols (polyalcohols), especially glycerol esters, (x) glycols, especially polyalkylene glycols, preferably polyethylene glycols, and the (poly)ethers and (poly)esters thereof, and also combinations of two or more of the aforementioned compounds.

Particularly preferably, component (c) or the emulsifier can be selected from the group of (i) fatty acids, fatty acid salts, fatty acid amides and fatty acid esters, especially fatty acid esters, and (ii) optionally (poly)ethoxylated and/or hydrogenated ricinus oils and also the combinations thereof.

Furthermore, it can be envisaged that the composition according to the invention also contains at least one chemical buffer system, especially in the form of buffer salt(s), as component (d).

In relation to the term chemical buffer or chemical buffer system, reference can be made in particular to RÖMPP Lexikon Chemie [RÖMPP chemical dictionary], 10th edition, Georg-Thieme-Verlag, Stuttgart/New York, volume 5, 1998, pages 3618/3619, keyword: “Buffer”, and to the literature referenced therein.

Within the scope of the present invention, the chemical buffer system, especially in the form of buffer salt(s), serves in particular to stabilize the composition according to the invention. More particularly, the setting of a constant pH by means of the buffer system surprisingly leads to there being efficient counteraction of an undesired degradation of the active ingredients, especially of components (a) and (b), during storage, even over a relatively long period. In this way, the shelf life of the composition according to the invention is improved.

The quantity of component (d) or chemical buffer system in the cineole-containing composition according to the present invention can vary or be adjusted within wide ranges. According to the invention, it is preferred when the composition according to the invention contains component (d) or the chemical buffer system, based on the composition and calculated as the sum total of all constituents of the chemical buffer system, in relative quantities within the range from 0.001 to 4% by weight, especially from 0.01 to 3% by weight, by preference from 0.05 to 2% by weight, preferably from 0.1 to 1.5% by weight, particularly preferably from 0.2 to 1% by weight.

Within the scope of the present invention, component (d) or the chemical buffer system serves in particular to set and/or to keep constant the pH of the composition according to the invention. As explained above, this leads to there being efficient counteraction of an undesired degradation of the active ingredients, especially of components (a) and (b), during storage, even over a relatively long period, and, in this way, to the shelf life of the composition according to the invention being improved.

Preferably according to the invention, it can be envisaged that component (d) or the chemical buffer system is present as a dihydrogen phosphate/monohydrogen phosphate buffer system (sometimes also synonymously referred to as “H₂PO₄⁻/HPO₄²⁻ buffer (system)” or “phosphate buffer (system)”), especially as an alkali metal dihydrogen phosphate/alkali metal monohydrogen phosphate buffer system, preferably having a dihydrogen phosphate/monohydrogen phosphate molar ratio of greater than 5:1, especially within the range from 5:1 to 110:1, particularly preferably within the range from 6:1 to 105:1, very particularly preferably within the range from 7:1 to 200:1, even more preferably within the range from 7:1 to 100:1. When using this specific buffer system, especially with simultaneous observance of the aforementioned molar ratios, there is counteraction to a particular extent of an undesired degradation of the active ingredients, especially of components (a) and (b), during storage, even over a relatively long period, and the shelf life of the composition according to the invention is improved to a particular extent.

The pH of the composition according to the invention can absolutely vary within wide ranges. According to the invention, it is preferred when the composition according to the invention has a pH within the range from 4.5 to 8.0, especially within the range from 5.0 to 6.5, by preference within the range from 5.0 to 6.2, preferably within the range from 5.0 to 6.0, even more preferably within the range from 5.1 to 6.0, very particularly preferably within the range from 5.2 to 5.9, most preferably within the range from 5.3 to 5.9, or when the pH of the composition according to the invention is set and/or kept constant within the range from 4.5 to 8.0, especially within the range from 5.0 to 6.5, by preference within the range from 5.0 to 6.2, preferably within the range from 5.0 to 6.0, even more preferably within the range from 5.1 to 6.0, very particularly preferably within the range from 5.2 to 5.9, most preferably within the range from 5.3 to 5.9 (preferably by means of at least one chemical buffer system, especially as defined above). With observance of the aforementioned pH ranges, there is counteraction to a particular extent of an undesired degradation of the active ingredients, especially of components (a) and (b), during storage, even over a relatively long period, and the shelf life of the composition according to the invention is also improved to a particular extent.

Within the scope of the present invention, the pH can be determined using methods known per se to a person skilled in the art. More particularly, the pH can be determined according to the method in accordance with Ph. Eur. [European Pharmacopoeia] 7th edition, basic edition 2011, section 2.2.3.

According to a particular embodiment of the present invention, it can be envisaged that the composition according to the invention also contains at least one preservative and/or disinfectant (antiseptic) as component (e).

A preservative and/or disinfectant (antiseptic) in the context of the present invention is a formulation excipient which serves to counteract microorganisms and/or to develop an antimicrobial or wound-disinfecting effect.

Within the scope of the present invention, it is intended through the use of component (e) to counteract an (excessive) infestation of the composition according to the invention with microorganisms and thus an undesired contamination. In this way, the stability of the resulting composition according to the invention, especially also with respect to a storage over relatively long periods, is improved. As has been found by the applicant, surprisingly, the presence of a preservative and/or disinfectant (antiseptic) also supports, however, the anti-inflammatory and antibacterial and antiviral activity profile of the cineole during the application of the composition according to the invention in the treatment of rhinitises.

The use of a preservative and/or disinfectant (antiseptic) with respect to the composition according to the invention is absolutely optional, but is a preferred embodiment for the aforementioned reasons.

The quantity of component (e) or preservative and/or disinfectant (antiseptic) in the cineole-containing composition according to the present invention can vary or be adjusted within wide ranges. More particularly, it can be envisaged that the composition according to the invention contains component (e) or the preservative and/or disinfectant (antiseptic), based on the composition, in relative quantities within the range from 0.001 to 10% by weight, especially from 0.005 to 5% by weight, by preference from 0.01 to 2% by weight, preferably from 0.01 to 1% by weight, particularly preferably from 0.01 to 0.5% by weight, even more preferably from 0.02 to 0.1% by weight. Nevertheless, it may be necessary, depending on the individual case or depending on the application, to deviate from the aforementioned values without departing from the scope of the present invention. This is at the discretion of a person skilled in the art.

Component (e) or the preservative and/or disinfectant (antiseptic) can absolutely be selected from a multiplicity of diverse compounds. According to a preferred embodiment, it can be envisaged that component (e) or the preservative and/or disinfectant (antiseptic) is selected from the group of (i) alkylbenzyldimethylammonium chlorides, especially C₈-C₁₈-alkylbenzyldimethylammonium chlorides, and mixtures of various alkylbenzyldimethylammonium chlorides, preferably benzalkonium chloride, (ii) polyhexanide, (iii) sorbic acid and the salts thereof (sorbates), especially potassium sorbate, (iv) chlorhexidine, (v) para-hydroxybenzoic acid esters and mixtures of various para-hydroxybenzoic acid esters, preferably alkyl 4-hydroxybenzoates, and also combinations of two or more of the aforementioned compounds, preferably from the group of (i) alkylbenzyldimethylammonium chlorides, especially C₈-C₁₈-alkylbenzyldimethylammonium chlorides, and mixtures of various alkylbenzyldimethylammonium chlorides, preferably benzalkonium chloride, (ii) polyhexanide, (iii) sorbic acid and the salts thereof (sorbates), especially potassium sorbate, and also combinations of two or more of the aforementioned compounds. Particularly preferably, component (e) or the preservative and/or disinfectant (antiseptic) is selected from the group of benzalkonium chloride, polyhexanide and sorbates (e.g., potassium sorbate) and also the combinations thereof. According to the invention, benzalkonium chloride (i.e., a mixture of alkylbenzyldimethylammonium chlorides, the alkyl part of which consists of C₈-C₁₈ chains) is very particularly preferred as component (e).

Furthermore, it can be envisaged that the composition according to the invention also contains at least one thickener as component (f).

A thickener in the context of the present invention is a formulation excipient which serves to increase the viscosity of the composition according to the invention (i.e., to make the composition more viscous or less flowable). Within the scope of the present invention, the intended achievement due to the use of component (f) is that the composition according to the invention during its intranasal application adheres longer at the nasal mucous membrane and in this way the duration of action is prolonged. More particularly, the so-called bioadhesion is improved in this way. Also, it is possible in this way to specifically control the release of active ingredient. Because of the mucoadhesive effect which is improved owing to the presence of the thickener, it is possible to achieve a longer residence time at the site of action and an improved moisturization of the nasal mucous membrane.

The quantity of component (f) or thickener in the cineole-containing composition according to the present invention can vary or be adjusted within wide ranges. More particularly, it can be envisaged that the composition according to the invention contains component (f) or the thickener, based on the composition, in relative quantities within the range from 0.0001 to 10% by weight, especially from 0.001 to 8% by weight, by preference from 0.005 to 5% by weight, preferably from 0.01 to 3% by weight, particularly preferably from 0.05 to 2% by weight, even more preferably from 0.1 to 1% by weight. Nevertheless, it may be necessary, depending on the individual case or depending on the application, to deviate from the aforementioned values without departing from the scope of the present invention. This is at the discretion of a person skilled in the art.

Component (f) or the thickener can absolutely be selected from a multiplicity of diverse compounds. According to a preferred embodiment, it can be envisaged that component (f) or the thickener is selected from the group of (i) preferably acidic glycosaminoglycans or the physiologically safe salts or derivatives thereof, especially hyaluronic acids or the physiologically safe salts thereof (hyaluronates), (ii) polysaccharides and hydrocolloids, especially carrageenans, (iii) celluloses and cellulose derivatives, especially cellulose esters and cellulose ethers, (iv) alginic acids and the salts thereof (alginates), (v) poly(meth)acrylic acids and poly(meth)acrylates, (vi) polyalkylene glycols, especially polyethylene glycols, (vii) xanthans and also combinations of two or more of the aforementioned compounds.

Particularly preferably, component (f) or the thickener is selected from the group of hyaluronic acids or the physiologically safe salts thereof (hyaluronates), carrageenans and also the combinations thereof.

If a cellulose or cellulose derivative is used as component (f) or as thickener, hydroxypropylmethylcellulose (also synonymously referred to as “HPMC”, “hypromellose”, etc.) or the derivatives thereof (especially ethers and/or esters) is used in particular. Hydroxypropylmethylcellulose (HPMC) is a substance mixture of various, partly alkyl-substituted celluloses. It is commercially available in various degrees of polymerization and different degrees of substitution.

Furthermore, it can be envisaged that the composition according to the invention also contains at least one ectoine or ectoine derivative, especially a hydroxyectoine, as component (g), especially in relative quantities within the range from 0.0001 to 10% by weight, especially from 0.001 to 5% by weight, by preference from 0.01 to 2% by weight, based on the composition.

Equally, it can be envisaged that the composition according to the invention also contains at least one preferably imidazoline-based alpha sympathomimetic or the physiologically safe salts thereof as component (h); this applies in particular when a decongestant or swelling-reducing effect of the composition according to the invention is additionally desired to a particular extent (even though the presence of an alpha sympathomimetic is absolutely not necessary for the therapeutic success of the composition according to the invention). In this embodiment, the preferably imidazoline-based alpha sympathomimetic can in particular be selected from xylometazoline or oxymetazoline, especially in the form of the physiologically safe salts thereof, particularly preferably in the form of the hydrochloride salts thereof, and be particularly preferably xylometazoline hydrochloride. In this embodiment, the composition according to the invention can contain the preferably imidazoline-based alpha sympathomimetic, based on the composition, in relative quantities within the range from 0.001 to 2% by weight, especially from 0.005 to 1.5% by weight, by preference from 0.01 to 1.2% by weight, preferably from 0.02 to 1.0% by weight, particularly preferably from 0.03 to 0.5% by weight, very particularly preferably from 0.04 to 0.2% by weight.

Furthermore, it can be envisaged that the composition according to the invention also contains sodium chloride, especially in relative quantities within the range from 0.001 to 5% by weight, especially from 0.01 to 2% by weight, by preference from 0.1 to 1% by weight, based on the composition. This can apply in particular when an additional moisturization of the nasal mucous membrane is intended.

According to a particular embodiment, the present invention provides a cineole-containing composition, especially a pharmaceutical composition, which is preferably suitable for topical application, especially nasal application, preferably intranasal application, especially for the treatment of rhinitises, especially a composition according to the present invention as described above,

wherein the composition contains, in combination and in effective, especially pharmaceutically effective, quantities in each case:
(a) cineole, preferably 1,8-cineole, especially in relative quantities within the range from 0.001 to 10% by weight, especially from 0.002 to 5% by weight, by preference from 0.01 to 3% by weight, preferably from 0.05 to 3% by weight, particularly preferably from 0.1 to 2.5% by weight, very particularly preferably from 0.2 to 2% by weight, even more preferably from 0.3 to 1% by weight, most preferably from 0.4 to 0.8% by weight;
(b) pantothenol, preferably dexpanthenol (D-pantothenol), or the physiologically safe esters thereof, especially in relative quantities within the range from 0.01 to 10% by weight, especially from 0.1 to 8% by weight, by preference from 0.5 to 7% by weight, preferably from 1 to 6.5% by weight, particularly preferably from 2 to 6% by weight, very particularly preferably from 4 to 6% by weight, even more preferably from 4.5 to 5.5% by weight;
(c) optionally at least one emulsifier, especially in relative quantities within the range from 0.0001 to 5% by weight, especially from 0.001 to 2.5% by weight, by preference from 0.005 to 2% by weight, preferably from 0.01 to 1.5% by weight, particularly preferably from 0.01 to 1% by weight, very particularly preferably from 0.01 to 0.5% by weight, even more preferably from 0.02 to 0.08% by weight;
(d) optionally at least one chemical buffer system, especially in the form of buffer salt(s), especially in relative quantities, calculated as the sum total of all constituents of the chemical buffer system, within the range from 0.001 to 4% by weight, especially from 0.01 to 3% by weight, by preference from 0.05 to 2% by weight, preferably from 0.1 to 1.5% by weight, particularly preferably from 0.2 to 1% by weight;
(e) optionally at least one preservative and/or disinfectant (antiseptic), especially in relative quantities within the range from 0.001 to 10% by weight, especially from 0.005 to 5% by weight, by preference from 0.01 to 2% by weight, preferably from 0.01 to 1% by weight, particularly preferably from 0.01 to 0.5% by weight, even more preferably from 0.02 to 0.1% by weight;
(f) optionally at least one thickener, especially in relative quantities within the range from 0.0001 to 10% by weight, especially from 0.001 to 8% by weight, by preference from 0.005 to 5% by weight, preferably from 0.01 to 3% by weight, particularly preferably from 0.05 to 2% by weight, even more preferably from 0.1 to 1% by weight;
(g) optionally at least one ectoine or ectoine derivative, especially a hydroxyectoine, especially in relative quantities within the range from 0.0001 to 10% by weight, especially from 0.001 to 5% by weight, by preference from 0.01 to 2% by weight;
(h) optionally at least one preferably imidazoline-based alpha sympathomimetic or the physiologically safe salts thereof, especially in relative quantities within the range from 0.001 to 2% by weight, especially from 0.005 to 1.5% by weight, by preference from 0.01 to 1.2% by weight, preferably from 0.02 to 1.0% by weight, particularly preferably from 0.03 to 0.5% by weight, very particularly preferably from 0.04 to 0.2% by weight;
(i) sodium chloride, especially in relative quantities within the range from 0.001 to 5% by weight, especially from 0.01 to 2% by weight, by preference from 0.1 to 1% by weight;
   with all aforementioned relative specified quantities being based on the composition.

The cineole-containing composition according to the present invention can absolutely exist in diverse outward forms and/or be developed in diverse formulation forms.

In general, the composition according to the present invention can be present as an aqueous composition. More particularly, the composition according to the present invention can be aqueous-based and/or be present in aqueously formulated form, especially in the form of an aqueous solution or aqueous solubilization. In this way, a good physiological compatibility and a good applicability are ensured.

According to a preferred embodiment, the cineole-containing composition according to the invention can be present as an aqueous system, especially as an aqueous single-phase system, preferably as an aqueous solution or aqueous solubilization. In general, the composition according to the present invention comprises a water-based excipient or vehicle. Preferably, the composition according to the invention is present as a clear, colorless aqueous solution. This ensures, firstly, a good physiological tolerance and a good applicability and, secondly, a good stability, especially storage stability, specifically even over long periods.

Within the scope of the present invention, the appearance of the composition according to the invention can be determined using methods known per se to a person skilled in the art. More particularly, the appearance of the composition according to the invention can be determined according to the method in accordance with Ph. Eur. [European Pharmacopoeia] 7th edition, basic edition 2011, sections 2.2.1 and 2.2.2.

The osmolality of the cineole-containing composition according to the present invention can too vary within wide limits. To ensure a good physiological compatibility and, at the same time, good applicability, it has been found to be useful when the composition according to the invention has an osmolality within the range from 300 to 600 mosm/kg, especially within the range from 310 to 550 mosm/kg, by preference within the range from 300 to 525 mosm/kg, preferably within the range from 325 to 510 mosm/kg, particularly preferably within the range from 350 to 500 mosm/kg. Nevertheless, it may be necessary, depending on the individual case or depending on the application, to deviate from the aforementioned values without departing from the scope of the present invention; this is at the free discretion of a person skilled in the art active in this field.

Within the scope of the present invention, the osmolality of the composition according to the invention can be determined using methods known per se to a person skilled in the art. More particularly, the osmolality of the composition according to the invention can be determined according to the method in accordance with Ph. Eur. [European Pharmacopoeia] 7th edition, basic edition 2011, section 2.2.35, chapter 5.3.9.

Likewise, the density of the cineole-containing composition according to the present invention can vary within wide ranges. More particularly, it can be envisaged that the cineole-containing composition according to the invention has, at a temperature of 20° C. and at a pressure of 1013.25 mbar (atmospheric pressure), a relative density, based on pure water, within the range from 1.001 to 1.2, especially within the range from 1.005 to 1.15, by preference within the range from 1.005 to 1.105. Nevertheless, it may be necessary, depending on the individual case or depending on the application, to deviate from the aforementioned values without departing from the scope of the present invention.

Within the scope of the present invention, the relative density of the composition according to the invention can be determined using methods known per se to a person skilled in the art. More particularly, the relative density of the composition according to the invention can be determined according to the method in accordance with Ph. Eur. [European Pharmacopoeia] 7th edition, basic edition 2011, section 2.2.5.

Depending on the application, the viscosity of the cineole-containing composition according to the present invention can also be adjusted or selected within wide ranges.

According to a particular embodiment in which the composition according to the invention is present in the absence of a thickener (i.e., when the composition according to the invention is formulated without a thickener or does not contain a thickener), the composition according to the present invention can, at a temperature of 20° C., have a dynamic viscosity within the range from 1.001 to 2 mPas, especially from 1.01 to 1.9 mPas, by preference from 1.05 to 1.8 mPas, particularly preferably from 1.1 to 1.5 mPas (it being possible to determine the dynamic viscosity especially according to the method in accordance with Ph. Eur. [European Pharmacopoeia] 7th edition, basic edition 2011, section 2.2.9 capillary viscometer [preferably Ubbelohde viscometer]). Such an embodiment is especially preferred when a good applicability of the composition by means of spray application is paramount.

According to a further, alternative particular embodiment in which the composition according to the invention is present in the presence of a thickener (i.e., when the composition according to the invention is formulated with a thickener or contains a thickener), the composition according to the present invention can, at a temperature of 20° C., have a dynamic viscosity of at least 1.5 mPas, especially within the range from 1.5 to 10⁵ mPas, by preference from 1.6 to 10⁴ mPas, particularly preferably from 1.7 to 10³ mPas, even more preferably from 2 to 100 mPas (it being possible to determine the dynamic viscosity especially according to the method in accordance with Ph. Eur. [European Pharmacopoeia] 7th edition, basic edition 2011, section 2.2.9 capillary viscometer [preferably Ubbelohde viscometer]). Such an embodiment is especially preferred when a prolonged duration of action and/or an improved bioadhesion of the composition is paramount.

Likewise, the refractive index of the cineole-containing composition according to the present invention can vary within wide ranges. More particularly, it can be envisaged that the cineole-containing composition according to the invention has, at a temperature of 20° C. and at a pressure of 1013.25 mbar (atmospheric pressure), a refractive index within the range from 1.10 to 1.50, especially within the range from 1.20 to 1.40, by preference within the range from 1.25 to 1.39. Nevertheless, it may be necessary, depending on the individual case or depending on the application, to deviate from the aforementioned values without departing from the scope of the present invention.

Within the scope of the present invention, the refractive index of the composition according to the invention can be determined using methods known per se to a person skilled in the art. More particularly, the refractive index of the composition according to the invention can be determined according to the method in accordance with Ph. Eur. [European Pharmacopoeia] 7th edition, basic edition 2011, section 2.2.6.

As already explained above, the cineole-containing composition according to the present invention has a good stability, especially storage stability and long-term stability. According to a particular embodiment, the composition is designed to be stable, especially storage-stable, for at least 6 months, especially at least 12 months, by preference at least 24 months, preferably at least 36 months, at temperatures within the range from 20° C. to 50° C., at a pressure of 1013.25 mbar (atmospheric pressure) and at a relative humidity within the range from 50% to 90%.

Furthermore, it can be envisaged according to the invention that the cineole-containing composition according to the invention comprises at least one further ingredient, especially excipient and/or additive. The further ingredient, especially excipient and/or additive, can in particular be selected from the group of processing aids, stabilizers, emulsifiers, antioxidants, humectants, thickeners, antiseptics, dyes, flavorings, odorants, fragrances, diluents, binders, wetting agents, vitamins, trace elements, minerals, micronutrients and/or essential oils and also the combinations thereof (i.e., combinations of two or more of the aforementioned ingredients).

As explained at the beginning, the cineole-containing composition according to the present invention has a broad application profile. More particularly, the present invention provides a cineole-containing composition as described above for use in the prophylactic and/or curative topical treatment of rhinitises, especially of Rhinitis acuta.

Furthermore, the present invention provides—according to a second aspect of the present invention—an applicator containing a cineole-containing composition according to the present invention as described above, the applicator according to the invention being especially suitable for topical application, especially nasal application, preferably intranasal application, and being present preferably in the form of a container having a dropping device or spray device.

According to the invention, it is preferred when the applicator according to the invention has a spray device for the preferably uniform application of the composition according to the present invention in a quantity per spray action within the range from 25 μl to 300 μl, especially within the range from 50 μl to 200 μl, by preference within the range from 75 μl to 125 μl.

In this connection, it is envisaged in particular that the applicator according to the invention has a container or a reservoir having a volume within the range from 5 ml to 100 ml, especially from 10 ml to 50 ml. Said container or said reservoir then serves to accommodate the composition according to the invention.

For further details relating to the applicator according to the invention, reference can be made to the preceding explanations in relation to the composition according to the invention, which apply accordingly to the applicator according to the invention.

The present invention further provides for—according to a third aspect of the present invention—the use of a cineole-containing composition according to the present invention as described above for the prophylactic and/or curative topical treatment of rhinitises of any type, especially of Rhinitis acuta, or the use of a composition according to the present invention as described above for the preparation of a medicament for the prophylactic and/or curative topical treatment of rhinitises of any type, especially of Rhinitis acuta.

The term medicament (also synonymously pharmaceutical), as used within the scope of the present invention, is to be understood in a very extensive manner and encompasses not only medicaments or pharmaceuticals as such (i.e., in respect of regulations covering medicaments), but also especially so-called medical devices and additionally, however, even homeopathics and dietary supplements as well as cosmetics and implements. In other words, the cineole-containing composition according to the invention can thus be present in the form of a medicament (pharmaceutical), medical device, homeopathic, dietary supplement, cosmetic or implement.

More particularly, the composition according to the invention can be used for treating rhinitises of any type, especially Rhinitis acuta, Rhinitis allergica, Rhinitis atrophicans, Rhinitis hyperplastica or hypertrophicans, Rhinitis mutilans, Rhinitis nervosa or vasomotorica, environmental rhinitis or Rhinitis pseudomembranacea, preferably Rhinitis acuta.

As described above, within the scope of the use according to the invention, the composition according to the invention, as described above, can be applied in a topical manner, especially in a nasal manner, preferably in an intranasal manner. In this case, use can be made in particular of the applicator according to the invention, as has been described above.

For further details relating to the use according to the invention, reference can be made to the preceding explanations in relation to the other aspects of the invention, which explanations apply accordingly to the use according to the invention.

Further designs, modifications and variants and also advantages of the present invention are readily identifiable and realizable for a person skilled in the art upon reading the description, without said person departing from the scope of the present invention.

The following exemplary embodiments merely serve to illustrate the present invention without, however, restricting the present invention thereto.

### Exemplary Embodiments

### 1. Examples of Preparation

General Preparation Instructions
The procedure for preparing, in each case, 1000 g of a clear aqueous solution of the composition according to the invention is performed in a manner known per se to a person skilled in the art: First of all, a defined quantity of purified water is initially charged at room temperature and ambient pressure into an appropriate glass vessel equipped with a stirrer and subsequently adjusted using a solution of the chosen buffer system to a predefined pH within the range between 5.3 and 5.9 (e.g., pH of approx. 5.5) with subsequent checking of the attained pH. Thereafter, the following quantities of the further active ingredients and further ingredients (1,8-cineole, dexpanthenol and also optionally, for example, emulsifier, preservative/disinfectant (antiseptic), thickener, etc.) that are specified in the recipe examples are added thereto, and the entirety is then dissolved by thorough stirring to give a clear solution. Subsequently, the entirety is topped up with further water to the final weight of 1000 g and stirred to give a homogeneous solution. Optionally, the solution is filtered across neutral cellulose filters. Finally, the pH is checked again. The other relevant specifications of the solution are too checked for the set or preselected ranges of values (e.g., osmolality; relative density; microbiological purity and sterility; ruling out of contaminants, especially degradation products of the ingredients and active ingredients; viscosity; appearance; refractive index). A portion of the solution obtained is lastly filled into 10 ml or 20 ml brown-glass narrow-neck bottles, which can be equipped with either a dropping pipette or a spray-dispensing pump; for the intended use, one to three drops or one to two spray actions can be added and filled in each nostril several times daily. Another portion of the solution obtained is used for stability tests.
The recipes specified below are prepared according to these general preparation instructions. All the recipes are storage-stable for 36 months.

### Recipe 1 (Data Per 1000 g of Composition)

| | Quantity/ | |
|---|---|---|
| Ingredient | % by weight | Quality |
| Cineole | 2.000 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.040 | Ph. Eur. |
| septic) (benzalkonium chloride) | | |
| Emulsifier (PEG-40-ethoxylated | 0.200 | Ph. Eur. |
| and hydrogenated ricinus oil) | | |
| Purified water | 91.880 | Ph. Eur. |

Viscosity (20° C.): 1.10-1.20 mPas

### Recipe 2 (Data Per 1000 g of Composition)

| | Quantity/ | |
|---|---|---|
| Ingredient | % by weight | Quality |
| Cineole | 2.000 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.040 | Ph. Eur. |
| septic) (benzalkonium chloride) | | |
| Emulsifier (PEG-40-ethoxylated | 4.000 | Ph. Eur. |
| and hydrogenated ricinus oil) | | |
| Purified water | 88.080 | Ph. Eur. |

Viscosity (20° C.): 1.20-1.25 mPas

### Recipe 3 (Data Per 1000 g of Composition)

| | Quantity/ | |
|---|---|---|
| Ingredient | % by weight | Quality |
| Cineole | 1.000 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.040 | Ph. Eur. |
| septic) (benzalkonium chloride) | | |
| Emulsifier (PEG-40-ethoxylated | 2.000 | Ph. Eur. |
| and hydrogenated ricinus oil) | | |
| Purified water | 91.080 | Ph. Eur. |

Viscosity (20° C.): 1.18 mPas

### Recipe 4 (Data Per 1000 g of Composition)

| | Quantity/ | |
|---|---|---|
| Ingredient | % by weight | Quality |
| Cineole | 0.100 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.040 | Ph. Eur. |
| septic) (benzalkonium chloride) | | |
| Emulsifier (PEG-40-ethoxylated | 0.200 | Ph. Eur. |
| and hydrogenated ricinus oil) | | |
| Purified water | 93.780 | Ph. Eur. |

Viscosity (20° C.): 1.15-1.20 mPas

### Recipe 5 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.040 | Ph. Eur. |
| septic) (benzalkonium chloride) | | |
| Emulsifier (PEG-40-ethoxylated | 1.200 | Ph. Eur. |
| and hydrogenated ricinus oil) | | |
| Purified water | 92.880 | Ph. Eur. |

Viscosity (20° C.): 1.10-1.22 mPas

### Recipe 6 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.040 | Ph. Eur. |
| septic) (benzalkonium chloride) | | |
| Emulsifier (PEG-40-ethoxylated | 1.200 | Ph. Eur. |
| and hydrogenated ricinus oil) | | |
| Flavoring with coldness imparter | 0.040 | Ph. Eur. |
| (water-soluble cooling flavors | | |
| or corresponding essential oils) | | |
| Purified water | 92.240 | Ph. Eur. |

Viscosity (20° C.): 1.10-1.22 mPas

### Recipe 7 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.040 | Ph. Eur. |
| septic) (benzalkonium chloride) | | |
| Emulsifier (PEG-40-ethoxylated | 1.200 | Ph. Eur. |
| and hydrogenated ricinus oil) | | |
| Flavoring with coldness imparter | 0.080 | Ph. Eur. |
| (water-soluble cooling flavors | | |
| or corresponding essential oils) | | |
| Purified water | 92.200 | Ph. Eur. |

Viscosity (20° C.): 1.10-1.22 mPas

### Recipe 8 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.040 | Ph. Eur. |
| septic) (benzalkonium chloride) | | |
| Emulsifier (undecylenamidopropyl | 0.100 | Ph. Eur. |
| betaine) | | |
| Purified water | 93.380 | Ph. Eur. |

Viscosity (20° C.): 1.10-1.22 mPas

### Recipe 9 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.100 | Ph. Eur. |
| septic) (polyhexanide) | | |
| Emulsifier (undecylenamidopropyl | 0.100 | Ph. Eur. |
| betaine) | | |
| Purified water | 93.320 | Ph. Eur. |

Viscosity (20° C.): 1.10-1.22 mPas

### Recipe 10 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.100 | Ph. Eur. |
| septic) (polyhexanide) | | |
| Emulsifier (undecylenamidopropyl | 0.100 | Ph. Eur. |
| betaine) | | |
| Thickener (poloxamer) | 5.000 | Ph. Eur. |
| Purified water | 88.320 | Ph. Eur. |

Viscosity (20° C.): 10³ mPas

### Recipe 11 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Emulsifier (undecylenamidopropyl | 0.100 | Ph. Eur. |
| betaine) | | |
| Vitamins A and C (micelle) | 1.000 | Ph. Eur. |
| Purified water | 92.420 | Ph. Eur. |

Viscosity (20° C.): 1.15-1.25 mPas

### Recipe 12 (Data Per 1000 g of Composition)

| | Quantity/ | |
|---|---|---|
| Ingredient | % by weight | Quality |
| Cineole (liposomes) | 0.600 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Emulsifier (undecylenamidopropyl | 0.100 | Ph. Eur. |
| betaine) | | |
| Purified water | 93.420 | Ph. Eur. |

Viscosity (20° C.): 1.20-1.30 mPas

### Recipe 13 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Emulsifier (undecylenamidopropyl | 0.100 | Ph. Eur. |
| betaine) | | |
| Thickener (sodium hyaluronate) | 0.020 | Ph. Eur. |
| Preservative/disinfectant (anti- | 0.020 | Ph. Eur. |
| septic) (potassium sorbate) | | |
| Purified water | 93.380 | Ph. Eur. |

Viscosity (20° C.): 5·10² mPas

### Recipe 14 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Emulsifier (undecylenamidopropyl | 0.100 | Ph. Eur. |
| betaine) | | |
| Thickener (carrageenan) | 0.400 | Ph. Eur. |
| Purified water | 93.020 | Ph. Eur. |

Viscosity (20° C.): 10³ mPas

### Recipe 15 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Xylometazoline hydrochloride | 0.050 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.050 | Ph. Eur. |
| septic) (benzalkonium chloride) | | |
| Emulsifier (poloxamer) | 1.250 | Ph. Eur. |
| Thickener (sodium alginate) | 1.500 | Ph. Eur. |
| Purified water | 90.670 | Ph. Eur. |

Viscosity (20° C.): 1.5·10³ mPas

### Recipe 16 (Data Per 1000 g of Composition)

| | Quantity/ | |
|---|---|---|
| Ingredient | % by weight | Quality |
| Cineole | 1.000 | Ph. Eur. |
| Dexpanthenol | 5.000 | Ph. Eur. |
| Oxymetazoline hydrochloride | 0.100 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.050 | Ph. Eur. |
| septic) (benzalkonium chloride) | | |
| Emulsifier (poloxamer) | 1.250 | Ph. Eur. |
| Purified water | 91.720 | Ph. Eur. |

Viscosity (20° C.): 1.25-1.35 mPas

### Recipe 17 (Data Per 1000 g of Composition)

| | Quantity/ | |
|---|---|---|
| Ingredient | % by weight | Quality |
| Cineole | 1.000 | Ph. Eur. |
| Sodium pantothenate | 5.000 | Ph. Eur. |
| Oxymetazoline hydrochloride | 0.100 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.050 | Ph. Eur. |
| septic) (benzalkonium chloride) | | |
| Emulsifier (poloxamer) | 1.250 | Ph. Eur. |
| Purified water | 91.720 | Ph. Eur. |

Viscosity (20° C.): 1.25-1.35 mPas

### Recipe 18 (Data Per 1000 g of Composition)

| | Quantity/ | |
|---|---|---|
| Ingredient | % by weight | Quality |
| Cineole | 1.000 | Ph. Eur. |
| Dexpanthenol | 5.000 | Ph. Eur. |
| Oxymetazoline hydrochloride | 0.050 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.050 | Ph. Eur. |
| septic) (benzalkonium chloride) | | |
| Emulsifier (poloxamer) | 1.250 | Ph. Eur. |
| Purified water | 91.770 | Ph. Eur. |

Viscosity (20° C.): 1.25-1.30 mPas

### Recipe 19 (Data Per 1000 g of Composition)

| | Quantity/ | |
|---|---|---|
| Ingredient | % by weight | Quality |
| Cineole | 1.000 | Ph. Eur. |
| Sodium pantothenate | 5.000 | Ph. Eur. |
| Oxymetazoline hydrochloride | 0.050 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.050 | Ph. Eur. |
| septic) (benzalkonium chloride) | | |
| Emulsifier (poloxamer) | 1.250 | Ph. Eur. |
| Purified water | 91.770 | Ph. Eur. |

Viscosity (20° C.): 1.30-1.35 mPas

### Recipe 20 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Xylometazoline hydrochloride | 0.100 | Ph. Eur. |
| Buffer system: | 0.853 | Ph. Eur. |
| potassium dihydrogen phosphate/ | 0.027 | |
| sodium monohydrogen phosphate | | |
| (dodecahydrate) | | |
| Preservative/disinfectant (anti- | 0.050 | Ph. Eur. |
| septic) (benzalkonium chloride) | | |
| Emulsifier (poloxamer) | 1.250 | Ph. Eur. |
| Thickener (sodium alginate) | 1.500 | Ph. Eur. |
| Purified water | 90.620 | Ph. Eur. |

Viscosity (20° C.): 1.5·10³ mPas

### Recipe 21 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.008 | Ph. Eur. |
| potassium dihydrogen phosphate/ | 0.100 | |
| sodium monohydrogen phosphate | | |
| (dodecahydrate) | | |
| Sodium chloride | 3.000 | Ph. Eur. |
| Emulsifier (undecylenamidopropyl | 0.100 | Ph. Eur. |
| betaine) | | |
| Purified water | 91.192 | Ph. Eur. |

Viscosity (20° C.): 1.30-1.35 mPas

### Recipe 22 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.008 | Ph. Eur. |
| potassium dihydrogen phosphate/ | 0.100 | |
| sodium monohydrogen phosphate | | |
| (dodecahydrate) | | |
| Sodium chloride | 0.900 | Ph. Eur. |
| Emulsifier (undecylenamidopropyl | 0.100 | Ph. Eur. |
| betaine) | | |
| Purified water | 93.292 | Ph. Eur. |

Viscosity (20° C.): 1.25-1.30 mPas

### Recipe 23 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.008 | Ph. Eur. |
| potassium dihydrogen phosphate/ | 0.100 | |
| sodium monohydrogen phosphate | | |
| (dodecahydrate) | | |
| Ectoine | 2.000 | Ph. Eur. |
| Emulsifier (undecylenamidopropyl | 0.100 | Ph. Eur. |
| betaine) | | |
| Purified water | 92.192 | Ph. Eur. |

Viscosity (20° C.): 1.35-1.40 mPas

### Recipe 24 (Data Per 1000 g of Composition)

| | Quantity/ | |
|---|---|---|
| Ingredient | % by weight | Quality |
| Cineole | 0.600 | Ph. Eur. |
| Dexpanthenol | 5.000 | Ph. Eur. |
| Buffer system: | 0.008 | Ph. Eur. |
| potassium dihydrogen phosphate/ | 0.100 | |
| sodium monohydrogen phosphate | | |
| (dodecahydrate) | | |
| Ectoine | 2.000 | Ph. Eur. |
| Emulsifier (undecylenamidopropyl | 0.100 | Ph. Eur. |
| betaine) | | |
| Xylometazoline hydrochloride | 0.100 | Ph. Eur. |
| Purified water | 92.092 | Ph. Eur. |

Viscosity (20° C.): 1.38-1.40 mPas

### 2. Stability Tests and Further Tests

a) Stability Behavior Depending on pH
In accordance with the above general preparation instructions, aqueous compositions according to the present invention are in each case prepared with 0.600% by weight of 1,8-cineole and 5.000% by weight of dexpanthenol (based in each case on the composition).
Using different quantities of buffer and, if necessary, different buffer systems, the pH of the compositions is varied in each case and the stability behavior tested (storage in each case at 25° C., at a pressure of 1013.25 mbar [atmospheric pressure] and at a relative humidity of 75%).
The following data refer to the maximum shelf life at the different specified pH values (requirement for stability: degradation of cineole 1%, based on cineole; degradation of dexpanthenol 1%, based on dexpanthenol):

| pH of composition | Shelf life | |
|---|---|---|
| 3.0 | 27 | months |
| 4.0 | 26 | months |
| 4.5 | 28.5 | months |
| 5.0 | 30.5 | months |
| 5.3 | >36 | months |
| 5.5 | >36 | months |
| 5.9 | >36 | months |
| 6.5 | 30.5 | months |
| 7.5 | 29.5 | months |
| 8.0 | 26.5 | months |
| 9.0 | 23 | months |

As shown by the tests, the best shelf life is achieved within a pH range from 5.0 to 6.5, especially from 5.3 to 5.9. In the case of these compositions, a phosphate-based pH buffer system (i.e., dihydrogen phosphate/monohydrogen phosphate buffer system or H₂PO₄⁻/HPO₄²⁻ buffer) as specified above is used.

b) Stability Behavior Depending on Further Ingredients
In accordance with the above general preparation instructions, aqueous compositions (“compositions A1 to A6”) according to the present invention are in each case prepared with 0.800% by weight of 1,8-cineole and 4.750% by weight of dexpanthenol (based in each case on the composition). In each case, a pH within the range from 5.3 to 5.9 is set using a phosphate-based pH buffer system as specified above (0.853% by weight of potassium dihydrogen phosphate and 0.027% by weight of sodium monohydrogen phosphate dodecahydrate, based in each case on the composition).
A first composition according to the invention (“composition A1”) does not contain any further ingredients in addition to the aforementioned ingredients. Further ingredients (i.e., emulsifier [polyethoxylated hydrogenated ricinus oil] and/or preservative/disinfectant [benzalkonium chloride] and/or thickener [sodium hyaluronate]) are additionally added to the other compositions according to the invention (“compositions A2 to A6”), specifically as specified as follows (composition A2: 1.250% by weight of emulsifier; composition A3: 0.050% by weight of preservative/disinfectant; composition A4: 1.250% by weight of emulsifier and 0.050% by weight of preservative/disinfectant; composition A5: 1.500% by weight of thickener; composition A6: 1.250% by weight of emulsifier, 0.050% by weight of preservative/disinfectant and 1.500% by weight of thickener).
The stability behavior of these compositions is tested under defined conditions (storage in each case at 25° C., at a pressure of 1013.25 mbar [atmospheric pressure] and at a relative humidity of 75%). The following data refer to the maximum shelf life (requirement for stability: degradation of cineole 1%, based on cineole; degradation of dexpanthenol 1%, based on dexpanthenol):

| Composition | Shelf life |
|---|---|
| A1 | 37.5 months |
| A2 | 40.5 months |
| A3 | 39.5 months |
| A4 | 44.5 months |
| A5 | 38.5 months |
| A6 | 45.5 months |

As shown by the tests, particularly good shelf lives are obtained when further ingredients based on emulsifiers, preservatives/disinfectants and/or thickeners are added to the compositions according to the invention in addition to the actual active ingredients (cineole and dexpanthenol).

c) Tests Relating to Antibacterial and Antiviral Activity Behavior
Compositions A1 to A6 according to the invention that are described in the above exemplary embodiment 2 b) are tested for their antibacterial and antiviral activity behavior. A further composition A7 which is, however, not according to the invention is tested, the noninventive composition A7 corresponding to composition A1 according to the invention, but with the cineole being replaced with a corresponding proportion of water.
The antibacterial activity of the compositions is tested in a plate diffusion test and in a dilution test series of 18 bacterial strains (including Staphylococcus aureus, Haemophilus influenzae, Moraxella catarrhalis, Streptococcus pneumoniae, Bacillus sp., Citrobacter sp., Escherichia coli, Klebsiella sp., Salmonella sp. and Vibrio cholera) (determination of the MIC concentrations [Minimal Inhibitory Concentrations]). Compositions A1 to A6 according to the invention exhibit a good antibacterial activity, with composition A6 exhibiting the best antibacterial activity, followed by compositions A4, A3, A2, A5 and A1 (in order of antibacterial efficacy); the noninventive composition A7 exhibits essentially no antibacterial activity.
The antiviral activity of the compositions is tested against IBV (Infectious Bronchitis Virus, IBV Gray strain) in a so-called MTT assay in Vero-E6 (African green monkey kidney cells) (determination of the IC₅₀ concentrations). Compositions A1 to A6 according to the invention exhibit a good antiviral activity (anti-IBV properties), with composition A6 exhibiting the best antiviral activity, followed by compositions A4, A3, A2, A5 and A1 (in order of antiviral efficacy); the noninventive composition A7 exhibits essentially no antiviral activity.

### 3. Application Observations and Clinical Investigations

Part A
Compositions A1 to A6 according to the invention that are described in the above exemplary embodiment 2 b) and also the noninventive composition A7 that is described in the above exemplary embodiment 2 c) are tested in clinical applications (randomized placebo-controlled double-blind studies).
12 test subjects at a time from a group of test subjects consisting of 96 patients that are aged between 18 and 77 years (31 male, 58 female) and each suffering from acute viral rhinitises (“cold”) are treated with compositions A1 to A7 during the period of their disease for 5 days, with compositions A1 to A7 being applied topically as a spray several times daily. Another 12 test subjects from the group of test subjects are therapied with a noninventive composition A8, the noninventive composition A8 containing no active ingredients (placebo control).
The assessment of obstruction of nasal breathing, of rhinorrhea (runny nose), of redness of the nasal mucous membrane, of dryness of the nasal mucous membrane, of scab formation, of progress of healing, and of tolerability are documented as target variables (target parameters).
All the compositions tested exhibit an excellent tolerance. With regard to the quickening of the progress of healing, composition A6 provides the best result, followed by compositions A4, A3, A2, A5 and A1 (in order of efficacy or of the quickening of the progress of healing); the noninventive compositions A7 and A8 essentially do not lead to a quickening of the progress of healing.
With regard to the other target variables (target parameters), i.e., assessment of obstruction of nasal breathing, assessment of rhinorrhea (runny nose) and assessment of redness of the nasal mucous membrane, the assessments are reported in the form of so-called scores (score values) in the following table for the various compositions (0=no manifestation, 1=low manifestation, 2=moderate manifestation, 3=strong manifestation and 4=very strong manifestation); a relatively high score value therefore indicates a relatively high activity of the disease or a relatively poor efficacy.

| | Composition | | | | | | | |
|---|---|---|---|---|---|---|---|---|
| | A1 | A2 | A3 | A4 | A5 | A6 | A7* | A8* |
| Obstruction | | | | | | | | |
| of nasal | | | | | | | | |
| breathing | | | | | | | | |
| Base value | 2.9 ± 0.6 | 2.9 ± 0.5 | 2.8 ± 0.6 | 2.8 ± 0.7 | 2.9 ± 0.5 | 2.9 ± 0.4 | 2.8 ± 0.5 | 2.9 ± 0.6 |
| 3 days | 1.9 ± 0.8 | 1.8 ± 0.6 | 1.7 ± 0.6 | 1.6 ± 0.5 | 1.9 ± 0.6 | 1.4 ± 0.5 | 2.5 ± 0.9 | 2.7 ± 0.9 |
| 5 days | 1.5 ± 0.8 | 1.3 ± 0.5 | 1.1 ± 0.4 | 1.0 ± 0.4 | 1.4 ± 0.6 | 0.8 ± 0.3 | 2.0 ± 0.8 | 2.3 ± 1.0 |
| Rhinorrhea | | | | | | | | |
| Base value | 3.1 ± 0.6 | 3.0 ± 0.5 | 2.9 ± 0.7 | 3.0 ± 0.4 | 3.2 ± 0.7 | 3.1 ± 0.6 | 2.9 ± 0.5 | 3.0 ± 0.7 |
| 3 days | 1.7 ± 0.7 | 1.4 ± 0.5 | 1.3 ± 0.4 | 1.2 ± 0.3 | 1.6 ± 0.5 | 1.0 ± 0.3 | 2.6 ± 0.8 | 2.7 ± 1.0 |
| 5 days | 1.0 ± 0.6 | 0.8 ± 0.4 | 0.7 ± 0.3 | 0.6 ± 0.3 | 0.9 ± 0.5 | 0.4 ± 0.1 | 2.0 ± 0.9 | 2.1 ± 1.1 |
| Redness of | | | | | | | | |
| nasal | | | | | | | | |
| mucous | | | | | | | | |
| membrane | | | | | | | | |
| Base value | 3.0 ± 0.5 | 3.2 ± 0.7 | 2.9 ± 0.6 | 2.9 ± 0.5 | 3.0 ± 0.4 | 3.1 ± 0.5 | 3.0 ± 0.4 | 3.0 ± 0.4 |
| 3 days | 1.7 ± 0.8 | 1.5 ± 0.6 | 1.4 ± 0.5 | 1.3 ± 0.4 | 1.6 ± 0.7 | 1.1 ± 0.4 | 2.0 ± 0.7 | 2.7 ± 1.1 |
| 5 days | 0.9 ± 0.6 | 0.8 ± 0.3 | 0.6 ± 0.2 | 0.5 ± 0.2 | 0.9 ± 0.5 | 0.3 ± 0.1 | 1.5 ± 0.6 | 2.2 ± 0.9 |
| 5 days | 1.3 ± 0.7 | 1.0 ± 0.4 | 0.9 ± 0.4 | 0.7 ± 0.3 | 1.1 ± 0.5 | 0.5 ± 0.2 | 1.6 ± 0.9 | 2.1 ± 1.0 |

*Not inventive

Overall, all the tested compositions A1 to A6 according to the present invention exhibit an excellent efficacy in the treatment of acute rhinitises. With regard to efficacy, composition A6 according to the invention provides the best result, followed by compositions A4, A3, A2, A5 and A1 according to the invention (in order of efficacy). By contrast, the noninventive compositions A7 and A8 have essentially no efficacy.
Furthermore, further clinical investigations and further randomized placebo-controlled double-blind studies confirm a synergism of the effects of the active ingredients (i.e., cineole and dexpanthenol) of the compositions according to the invention, and this leads to a more clinically distinct or significant improvement in the treatment of rhinitises, specifically going markedly beyond the extent of the individual active ingredients.

Part B
In addition, two further compositions, each containing a decongestant, are prepared and tested:
In accordance with the above general preparation instructions, two aqueous compositions (“compositions A9 and A10”) are prepared: Composition A9 according to the invention contains 0.650% by weight of 1,8-cineole and 5.250% by weight of dexpanthenol and also, in addition, 0.050% by weight of xylometazoline hydrochloride as decongestant, 1.500% by weight of emulsifier and 0.065% by weight of preservative/disinfectant (based in each case on the composition), with a pH of about 5.5 being set using a phosphate-based pH buffer system as specified above (0.853% by weight of potassium dihydrogen phosphate and 0.027% by weight of sodium monohydrogen phosphate dodecahydrate, based in each case on the composition). The noninventive composition A10 corresponds to composition A9, but with the difference that, firstly, the cineole portion is completely replaced with water (i.e., the noninventive composition A10 does not contain 1,8-cineole) and, secondly, the xylometazoline hydrochloride portion is doubled (i.e., the xylometazoline hydrochloride portion is 0.100% by weight, based on the composition).
Compositions A9 and A10 are tested in clinical applications (randomized placebo-controlled double-blind studies): 11 test subjects at a time from a group of test subjects consisting of 22 patients that are aged between 19 and 79 years (10 male, 12 female) and each suffering from acute viral rhinitises (“cold”) are treated with compositions A9 and A10 during the period of their disease for 5 days, with the compositions being applied topically as a spray several times daily. The assessment of obstruction of nasal breathing, of rhinorrhea (runny nose), of redness of the nasal mucous membrane, of dryness of the nasal mucous membrane, of scab formation, of progress of healing, and of tolerability are documented as target variables (target parameters).
With regard to the target variables (target parameters) of obstruction of nasal breathing, which indicates the decongestant effect, score values of (1.0±0.3) and (0.5±0.2) are achieved after 3 and 5 days, respectively, for composition A9 according to the invention (base value: 3.1±0.6), whereas the score values are (1.2±0.4) and (0.7±0.3) after 3 and 5 days, respectively, for the noninventive composition A10 despite twice the quantity of decongestant (base value: 3.0±0.4).
This shows that, surprisingly, the presence of the cineole brings about a synergistic interaction with the decongestant, significantly increasing the effect of the decongestant. In this way, the quantity of decongestant can be reduced with a simultaneous increase in effect, and this leads to a significant improvement in the profile of adverse effects.

1-15. (canceled)

16. A cineole-containing pharmaceutical composition for the topical treatment of rhinitis,
wherein the composition comprises, in combination and in effective quantities, the following compounds:
(a) cineole, wherein the cineole is present as a pure substance and wherein the cineole is free of other terpenes,
wherein the composition comprises component (a), based on the composition, in relative quantities within the range from 0.001 to 10% by weight;
and
(b) at least one compound selected from the group consisting of (b1) pantothenol and physiologically tolerable esters thereof, (b2) pantothenic acid and physiologically tolerable salts thereof, and mixtures of (b1) and (b2), wherein the composition comprises component (b), based on the composition, in relative quantities within the range from 0.01 to 10% by weight;
wherein the composition has a pH within the range from 5.0 to 6.5.

17. The composition as claimed in claim 16,
wherein the composition comprises 1,8-cineole as component (a) and dexpanthenol as component (b)

18. The composition as claimed in claim 16,
wherein the cineole has a purity of at least 95% by weight, based on the cineole.

19. The composition as claimed in claim 16,
wherein the cineole has a purity of at least 99% by weight, based on the cineole.

20. The composition as claimed in claim 16,
wherein the composition comprises component (a), based on the composition, in relative quantities within the range from 0.002 to 5% by weight.

21. The composition as claimed in claim 16,
wherein component (a) is present in a liposome-surrounded form or in a liposomally packaged form.

22. The composition as claimed in claim 16,
wherein the composition comprises component (b), based on the composition, in relative quantities within the range from 0.1 to 8% by weight.

23. The composition as claimed in claim 16,
wherein the composition comprises components (a) and (b) in a quantitative weight ratio of component (a) to component (b) within the range of from 1:1 to 1:500.

24. The composition as claimed in claim 16,
wherein the composition further comprises at least one emulsifier as component (c).

25. The composition as claimed in claim 24,
wherein the composition comprises component (c), based on the composition, in relative quantities within the range from 0.0001 to 10% by weight.

26. The composition as claimed in claim 24,
wherein component (c) is selected from the group consisting of (i) fatty acids, fatty acid salts, fatty acid amides and fatty acid esters, (ii) optionally (poly)ethoxylated and/or hydrogenated castor oils, (iii) organic carboxylic acids and salts and esters thereof, (v) organic ammonium compounds, (vi) physiologically tolerably ionic or nonionic surfactants, (vii) phosphorlipids, (viii) ethylene oxide-propylene oxide block copolymers, (ix) esters of polyhydric alcohols, (x) glycols, and combinations of two or more of the aforementioned compounds.

27. The composition as claimed in claim 16,
wherein the composition further comprises at least one chemical buffer system as component (d).

28. The composition as claimed in claim 27,
wherein the composition comprises component (d), based on the composition and calculated as the sum total of all constituents of the chemical buffer system, in relative quantities within the range from 0.001 to 4% by weight.

29. The composition as claimed in claim 27,
wherein component (d) comprises a dihydrogen phosphate/monohydrogen phosphate buffer system.

30. The composition as claimed in claim 16,
wherein the composition also comprises at least one preservative or disinfectant as component (e).

31. The composition as claimed in claim 16,
wherein the composition also comprises at least one thickener as component (f).

32. The composition as claimed in claim 16,
wherein the composition further comprises at least one ectoine or ectoine derivative as component (g).

33. The composition as claimed in claim 16,
wherein the composition further comprises at least one imidazoline-based alpha sympathomimetic or a physiologically tolerable salt thereof.

34. A cineole-containing pharmaceutical composition for the topical treatment of rhinitis,
wherein the composition comprises, in combination and in effective quantities, the following compounds:
(a) 1,8-cineole in relative quantities within the range from 0.001 to 10% by weight, wherein the 1,8-cineole is present as a pure substance and wherein the cineole is free of other terpenes;
(b) dexpanthenol in relative quantities within the range from 0.01 to 10% by weight;
(c) at least one emulsifier in relative quantities within the range from 0.0001 to 5% by weight;
(d) at least one chemical buffer system, in relative quantities, calculated as the sum total of all constituents of the chemical buffer system, within the range from 0.001 to 4% by weight;
wherein all aforementioned relative specified quantities are based on the composition;
wherein the composition has a pH within the range from 5.0 to 6.5.

35. A method of treating a patient suffering from rhinitis,
wherein the method comprises the topical administration of a cineole-containing pharmaceutical composition in an efficient amount,
wherein the cineole-containing composition comprises, in combination and in effective quantities, the following compounds:
(a) cineole, wherein the cineole is present as a pure substance and wherein the cineole is free of other terpenes,
wherein the composition comprises component (a), based on the composition, in relative quantities within the range from 0.001 to 10% by weight;
and
(b) at least one compound selected from the group consisting of (b1) pantothenol and physiologically tolerable esters thereof, (b2) pantothenic acid and physiologically tolerable salts thereof, and mixtures of (b1) and (b2), wherein the composition comprises component (b), based on the composition, in relative quantities within the range from 0.01 to 10% by weight;
wherein the composition has a pH within the range from 5.0 to 6.5.

36. A method of treating a patient suffering from rhinitis,
wherein the method comprises the topical administration of a cineole-containing pharmaceutical composition in an efficient amount,
wherein the cineole-containing composition comprises, in combination and in effective quantities, the following compounds:
(a) 1,8-cineole in relative quantities within the range from 0.001 to 10% by weight, wherein the 1,8-cineole is present as a pure substance and wherein the cineole is free of other terpenes;
(b) dexpanthenol in relative quantities within the range from 0.01 to 10% by weight;
(c) at least one emulsifier in relative quantities within the range from 0.0001 to 5% by weight;
(d) at least one chemical buffer system, in relative quantities, calculated as the sum total of all constituents of the chemical buffer system, within the range from 0.001 to 4% by weight;
wherein all aforementioned relative specified quantities are based on the composition;
wherein the composition has a pH within the range from 5.0 to 6.5.

37. An applicator for topical intranasal application, wherein the applicator is in the form of a container comprising a dropping or spraying device and comprises a cineole-containing pharmaceutical composition,
wherein the cineole-containing composition comprises, in combination and in effective quantities, the following compounds:
(a) cineole, wherein the cineole is present as a pure substance and wherein the cineole is free of other terpenes,
wherein the composition comprises component (a), based on the composition, in relative quantities within the range from 0.001 to 10% by weight;
and
(b) at least one compound selected from the group consisting of (b1) pantothenol and physiologically tolerable esters thereof, (b2) pantothenic acid and physiologically tolerable salts thereof, and mixtures of (b1) and (b2), wherein the composition comprises component (b), based on the composition, in relative quantities within the range from 0.01 to 10% by weight;
wherein the composition has a pH within the range from 5.0 to 6.5.
